(19) United States
(12) Patent Application Publication
(10) Pub. No.: US 2014/0017264 A1
(43) Pub. Date: Jan. 16, 2014

(54) DOSAGE AND ADMINISTRATION OF BISPECIFIC SCFV CONJUGATES

(71) Applicants: Charlotte McDonagh, Cambridge, MA (US); Francis Gibbons, Cambridge, MA (US); Victor Moyo, Cambridge, MA (US)

(72) Inventors: Charlotte McDonagh, Cambridge, MA (US); Francis Gibbons, Cambridge, MA (US); Victor Moyo, Cambridge, MA (US)

(73) Assignee: MERRIMACK PHARMACEUTICALS, INC., Cambridge, MA (US)

(21) Appl. No.: 13/992,460

(22) PCT Filed: Dec. 12, 2011
(86) PCT No.: PCT/US11/64496

§ 371 Date: Sep. 5, 2013

Related U.S. Application Data
(60) Provisional application No. 61/421,992, filed on Dec. 10, 2010.

(51) Int. Cl. A61K 47/48

(52) U.S. Cl. CPC A61K 47/48338
USPC 424/178.1; 530/391.7

(57) ABSTRACT

Disclosed are methods for the therapeutic administration of bispecific scFv conjugates as single doses at at least weekly intervals. In certain embodiments the conjugate is MM-111 and is administered at intervals of every two weeks or every three weeks. In other embodiments a single loading dose of MM-111 is administered to a human patient followed at at least weekly intervals by a least a single maintenance dose of MM-111. The loading dose is larger than the maintenance dose.

### FIELD OF THE INVENTION

Provided are methods for the administration of therapeutic bispecific scFv conjugates in which a mutated human serum albumin linker is covalently bonded to distinct amino and carboxy terminal single-chain Fv molecules (scFvs).

### BACKGROUND OF THE INVENTION

Antibody-like binding moieties (including those in intact antibodies, antibody fragments, and engineered antibody fragments such as scFvs) are often used for therapeutic applications. Antibody fragments such as scFvs generally exhibit shorter serum half lives than intact antibodies, and in some therapeutic applications increased in vivo half lives would be desirable for therapeutic agents possessing the functionality of such fragments/scFvs.

Human serum albumin (HSA) is a protein of about 66,500 kD and is comprised of 585 amino acids including at least 17 disulphide bridges. As with many of the members of the albumin family, human serum albumin plays an important role in human physiology and is located in virtually every human tissue and bodily secretion. HSA has the ability to bind and transport a wide spectrum of ligands throughout the circulatory system, including the long-chain fatty acids, which are otherwise insoluble in circulating plasma. A bispecific scFv HSA conjugate designated MM-111 (also referred to as B2B3-1) is described in copending U.S. patent application Ser. No. 12/757,801, and PCT publication number WO2009/126920, each of which is incorporated herein by reference in its entirety. MM-111 is currently undergoing clinical trials, including an open-label Phase 1-2 and pharmacologic study of MM-111 in patients with advanced, refractory HER2 positive cancers, and an open-label Phase 1-2 trial of MM-111 in combination with trastuzumab (HERCEPTIN) in patients with advanced HER2 positive breast cancer. The ErbB2/ErbB3 (ErbB2/3) oncogenic heterodimer is the most potent ErbB receptor pairing with respect to strength of interaction, impact on receptor tyrosine phosphorylation, and effects on downstream signaling through mitogen activated protein kinase and phosphoinositide-3 kinase pathways. ErbB3 signaling has become recognized as an important mechanism of resistance to ErbB2 (HER-2) targeted agents (such as trastuzumab) in clinical use. In ErbB2 HIGH disease states resistance to directed therapies is driven by heregulin/ErbB3 signaling. Current ErbB2-targeted therapies do not effectively inhibit heregulin activated ErbB2/3. Preclinically combinations of MM-111 (inhibiting heregulin activation of ErbB2/3 without blocking ErbB2) with trastuzumab (targeting ErbB2) provide complete inhibition.

MM-111 specifically targets the ErbB2/ErbB3 heterodimer and abrogates ligand binding. In preclinical models of HER-2+ gastric, breast, ovarian and lung cancers, MM-111 inhibits ligand-induced ErbB3 phosphorylation, cell cycle progression, and tumor growth.

### SUMMARY OF THE INVENTION

Provided are methods of administering bispecific scFv conjugates in which an bispecific scFv is covalently bonded to amino and carboxy terminal binding moieties that are first and second single-chain Fv molecules (scFvs). An exemplary bispecific scFv conjugate of this type is MM-111 (B2B3-1). MM-111 is a polypeptide comprising the amino acid sequence set forth in SEQ ID NO:1. MM-111 comprises first and second binding moieties that are single-chain Fv molecules: the first binding moiety specifically binds ErbB3 and the second binding moiety specifically binds ErbB2. Dosage units comprising fixed amounts of MM-111 are also provided. In accordance with the invention, bispecific scFv conjugates such as MM-111 are administered at at least weekly intervals (e.g., weekly or biweekly) at a dose of at least 20 mg/kg. In certain embodiments an initial loading dose that is equal to or greater than 120% of the weekly or biweekly dose is administered at the onset of therapeutic treatment with the bispecific scFv conjugate.

### BRIEF DESCRIPTION OF THE DRAWINGS

FIG. 1 shows MM-111 serum concentration levels obtained in patients treated with 3, 6, 12, or 20 mg/kg of MM-111. A preclinical PK/PD model relating [drug] to tumor growth inhibition was used to select the EC80 as a target clinical trough level (Cmin). A clinical population-PK model indicates that a 20 mg/kg maintenance dose reaches or exceeds this target in 80% of the patients by week 3 of treatment. A loading dose of 25 mg/kg is predicted to achieve the target in week 1. X Axis =Serum concentration (mg/L). Y axis =Weeks 0, 2, 4, and 6.

FIG. 2 shows a clinical trial enrollment and response summary. FIG. 3 shows MM-111 cardiac safety data in the form of a graph showing changes in mid-ejection fraction (n=8) as determined from ECGs. No clinically significant abnormalities were observed.

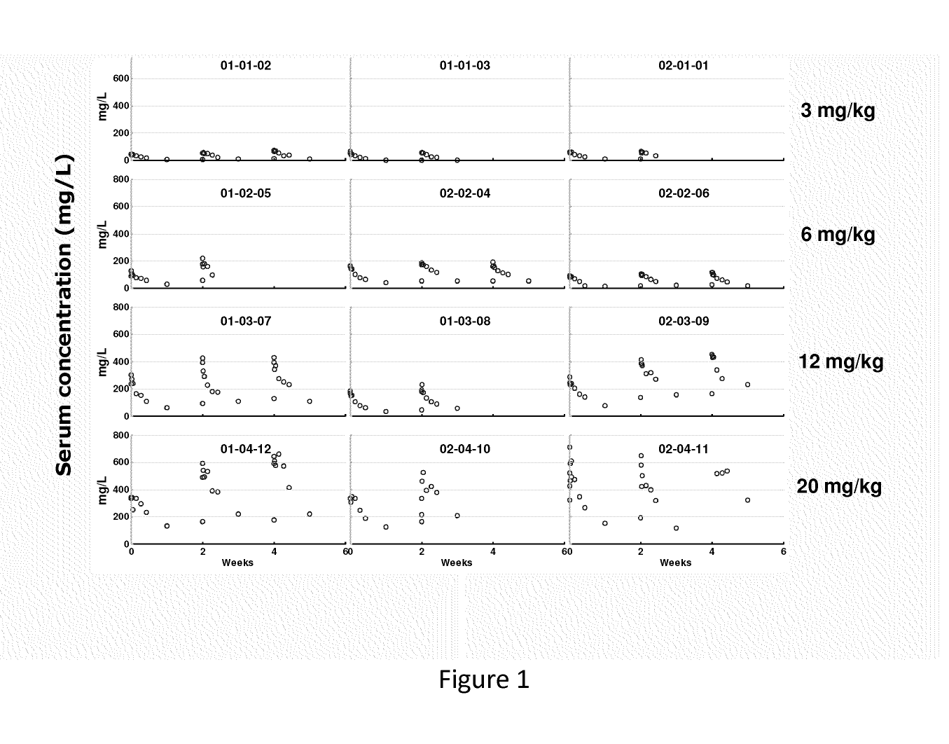

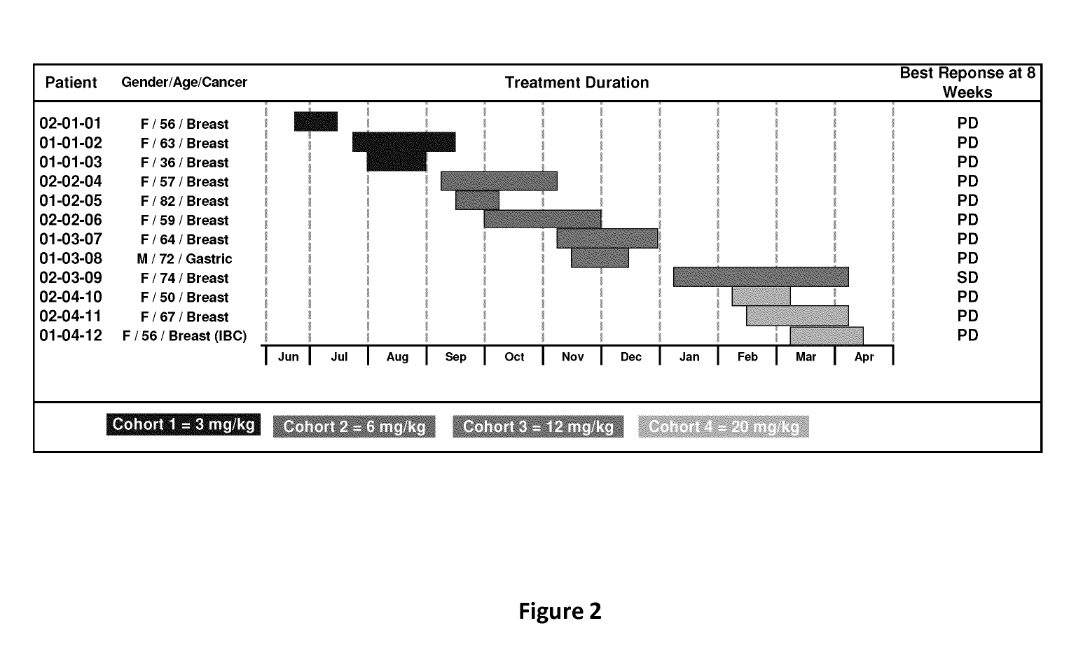

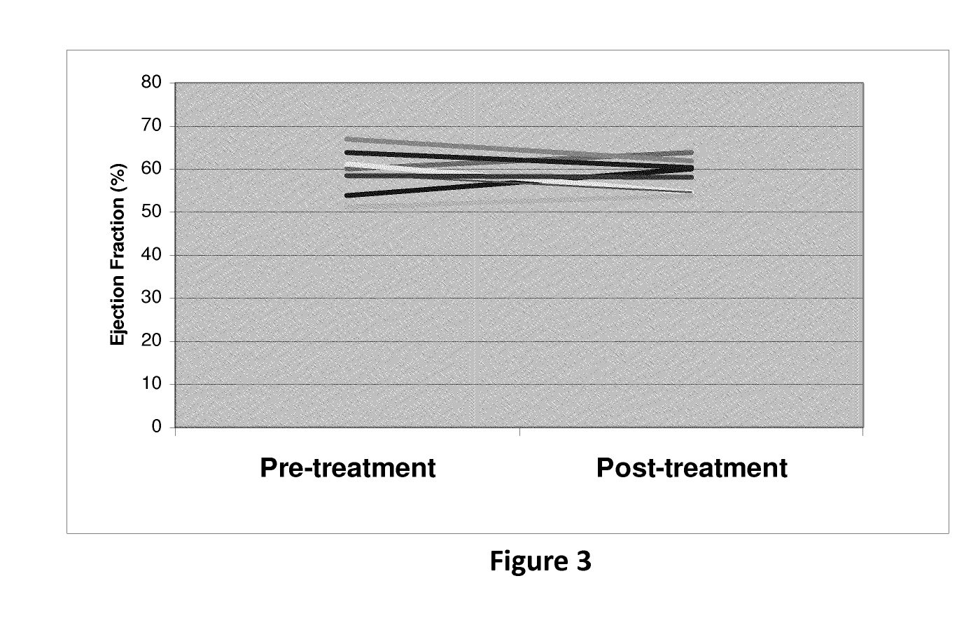

### DETAILED DESCRIPTION

### Methods and Compositions

Provided are methods of administering MM-111.

In certain aspects a first method is provided for the treatment of a human patient diagnosed with cancer characterized by expression of ErbB2 receptor, comprising administering an effective amount MM-111 to the patient at an interval measured in days, the method comprising: administering to the patient a single loading dose of at least 20 mg/kg of MM-111 followed at at least seven day intervals by at least one administration of a single maintenance dose of MM-111, wherein the maintenance dose is smaller than the loading dose. In other aspects the preceding method is one wherein the maintenance dose is at least 5 mg/kg less than the loading dose. In other aspects the preceding methods are methods wherein the at least seven day intervals are intervals of every 10 days. In other aspects the preceding methods are methods wherein the at least seven day intervals are intervals of every 14 days. In other aspects the preceding methods are methods wherein the at least seven day intervals are intervals of every 18 days. In other aspects the preceding methods are methods wherein the at least seven day intervals are intervals of every 21 days.

In certain aspects the first method is one wherein the at least seven day intervals are intervals of a number of days indicated by a top number, the loading dose of at least 20 mg/kg of MM-111 is a dose in mg/kg indicated by a middle number, and the maintenance dose of MM-111 smaller than the loading dose is a dose in mg/kg indicated by a bottom number in a cell of Table 1C selected from any populated cell of Table 1 C.

In certain aspects a second method is provided for the treatment of a human patient diagnosed with cancer characterized by expression of ErbB2 receptor, comprising administering an effective amount MM-111 to the patient, the method comprising: administering to the patient a single initial dose of at least 15 mg/kg of MM-111 followed at at least seven day intervals by at least one administration of a subsequent dose of MM-111 which is the same as the initial dose. In other aspects the preceding second method is one wherein the dose is about 20 mg/kg. In other aspects the preceding second method is one wherein the dose is about 30 mg/kg. In other aspects the preceding second method is one wherein the dose is about 44 mg/kg. In other aspects the preceding second method is one wherein the dose is about 75 mg/kg. In other aspects the preceding second method is one wherein the dose is about 105 mg/kg.

In certain aspects the second method is one wherein the at least seven day intervals are intervals of a number of days indicated by a top number and the dose of at least 15 mg/kg of MM-111 is a dose in mg/kg indicated by a bottom number in a cell of Table 1C selected from any populated cell of Table 1C.

In certain aspects a composition A is provided for use in the treatment of a human patient diagnosed with cancer characterized by expression of ErbB2 receptor, said composition comprising MM-111 for administration to the patient at an interval measured in days, as a single loading dose of at least 20 mg/kg of MM-111 followed at at least seven day intervals by at least one administration of a single maintenance dose of MM-111, wherein the maintenance dose is smaller than the loading dose. In certain aspects a composition B is provided which is composition A wherein the maintenance dose is at least 5 mg/kg less than the loading dose. In certain aspects composition A or composition B is one wherein the at least seven day intervals are intervals of every 10 days. In certain aspects composition A or composition B is one wherein the at least seven day intervals are intervals of every 14 days.

In certain aspects composition A or composition B is one wherein the at least seven day intervals are intervals of every 18 days. In certain aspects composition A or composition B is one wherein the at least seven day intervals are intervals of every 21 days.

In certain aspects composition A is a composition wherein the at least seven day intervals are intervals of a number of days indicated by a top number, the loading dose of at least 20 mg/kg of MM-111 is a dose in mg/kg indicated by a middle number, and the maintenance dose of MM-111 smaller than the loading dose is a dose in mg/kg indicated by a bottom number in a cell of Table 1C selected from any populated cell of Table 1C.

In an additional aspect, a composition C is provided for use in the treatment of a human patient diagnosed with cancer characterized by expression of ErbB2 receptor, said composition comprising MM-111 for administration to the patient at an interval measured in days as a single initial dose of at least 15 mg/kg of MM-111 followed at at least seven day intervals by at least one administration of a subsequent dose of MM-111 which is the same as the initial dose. In an additional aspect of composition C, the dose is about 20 mg/kg. In an additional aspect of composition C, the dose is about 30 mg/kg. In an additional aspect of composition C, the dose is about 44 mg/kg. In an additional aspect of composition C, the dose is about 75 mg/kg. In an additional aspect of composition C, the dose is about 105 mg/kg. In an additional aspect of composition C, the at least seven day intervals are intervals of a number of days indicated by a top number and the dose of at least 15 mg/kg of MM-111 is a dose in mg/kg indicated by a bottom number in a cell of Table 1C selected from any populated cell of Table 1 C.

### Kits and Unit Dosage Forms

Further provided are kits that include a pharmaceutical composition containing MM-111 including a pharmaceutically-acceptable carrier, in a therapeutically effective amount adapted for use in the preceding methods. The kits include instructions to allow a practitioner (e.g., a physician, nurse, or patient) to administer the composition contained therein to treat an ErbB2 expressing cancer.

Preferably, the kits include multiple packages of the single-dose pharmaceutical composition(s) containing an effective amount of MM-111 for a single administration in accordance with the methods provided above. Optionally, instruments or devices necessary for administering the pharmaceutical composition(s) may be included in the kits. For instance, a kit may provide one or more pre-filled syringes containing an amount of MM-111 that is about 100 times the dose in mg/kg indicated for administration in the above methods. Such unit dosage forms preferably contain about 2g, about 3 g, about 4.4 g, about 7.5 g or about 10.5 g.

Furthermore, the kits may also include additional components such as instructions or administration schedules for a patient suffering from a disease or condition (e.g., a cancer, autoimmune disease, or cardiovascular disease) to use the pharmaceutical composition(s) containing an bispecific scFv, or any binding, diagnostic, and/or therapeutic agent conjugated thereto.

It will be apparent to those skilled in the art that various modifications and variations can be made in the compositions, methods, and kits of the present invention without departing from the spirit or scope of the invention. Thus, it is intended that the present invention cover the modifications and variations of this invention provided they come within the scope of the appended claims and their equivalents.

### EXAMPLES

The following examples are provided by way of illustration only and not by way of limitation. Those of skill in the art will readily recognize a variety of non-critical parameters that could be changed or modified to yield essentially the same or similar results.

Example 1

### Mode of Administration of MM-111

MM-111 is prepared as a formulation containing 25 mg/ml MM-111 in a sterile aqueous solution comprising 20 mM L-histidine hydrochloride, 150 mM sodium chloride, pH 6.5, which is stored at 2-8° C.

MM-111 must be brought to room temperature prior to administration. Containers (e.g., vials) of MM-111 must not be shaken. The appropriate quantity of MM-111 is removed from the container, diluted in 250 mL of 0.9% normal saline and administered as an infusion using a low protein binding in-line filter (preferably a 0.22 micrometer filter).

MM-111 is initially administered over about 90 minutes (first administration). In the absence of an infusion reaction, subsequent doses are administered over about 60 minutes.

A patient's body weight at the start of a dosing cycle is to be used to calculate the dose used throughout the cycle. Should a patient's body weight change by more than 10%, a new total dose is calculated to reflect this change.

### Example 2

### Dosage and Administration of MM-111

Preferred plasma concentrations of MM111 achieved during treatment are at least 106 mg/L. It has now been discovered that certain combinations of dose frequency and dosage will achieve and maintain this plasma concentration during the course of treatment in at least half, and preferably in more than 60%, 70% or 80% of treated patients.

In certain embodiments a higher initial dose (loading dose-LD) is given, followed as defined intervals by at least one maintenance dose (MD). Intervals of dosing in days are typically indicated as QxD, wherein x represents an integer, so that a QxD of 7 indicates dosing every 7 days. Table 1A, Table 1B, and Table 1C below show doses and dosing intervals of the invention. In Table 1A, Table 1B, and Table 1C the indicated loading doses are optional—initial doses are preferably made at the indicated loading dose (LD), but may (e.g., as directed or at the physician's discretion) be made at the maintenance dose (MD). Table 1A provides a set of exemplary dosing intervals, loading doses and maintenance doses. Table 1B provides a variation of Table 1A allowing for dosage variability (indicated as “about”) of up to +/−3 mg/mL. Table 1C appears below and provides a more extensive set of exemplary dosing intervals, loading doses and maintenance doses. In each cell of Table 1A, Table 1B, and Table 1C, the top figure is the integer x in the interval QxD (e.g., 18 as the top figure in a cell indicates a dosing interval of Q18D or every 18 days), the middle figure represents the (optional) loading dose (LD) in mg/kg, and the bottom figure represents the maintenance dose (MD) in mg/kg. Thus the top cell in Table 1 A indicates a dosing interval (QxD) of once every seven days, a loading dose (optional) of 25 mg per kg of patient body weight, and a maintenance dose of 20 mg per kg of patient body weight; while the cell furthest to the right on the top row of Table 1C indicates a dosing interval (QxD) of once every seven days, a loading dose (optional) of 30 mg per kg of patient body weight, and a maintenance dose of 15 mg per kg of patient body weight.

TABLE 1A
7
25
20
7
40
30
14
60
45
14
90
75
21
120
105

```
TABLE 1B
  7
about 25
about 20
  7
about 40
about 30
14
about 60
about 44
14
about 90
about 75
21
  about 120
  about 105
```

TABLE 1C

| | | | | | | | | | | | | |
|---|---|---|---|---|---|---|---|---|---|---|---|---|
| 7 | 7 | 7 | 7 | 7 | 7 | 7 | 7 | 7 | 7 | 7 | 7 | 7 |
| 10 | 15 | 20 | 25 | 30 | 15 | 20 | 25 | 30 | 35 | 20 | 25 | 30 |
| 5 | 5 | 5 | 5 | 5 | 10 | 10 | 10 | 10 | 10 | 15 | 15 | 15 |
| 7 | 7 | 7 | 7 | 7 | 7 | 7 | 7 | 7 | 7 | 7 | 7 | 7 |
| 35 | 40 | 25 | 30 | 35 | 40 | 45 | 30 | 35 | 40 | 45 | 50 | 55 |
| 15 | 15 | 20 | 20 | 20 | 20 | 20 | 25 | 25 | 25 | 25 | 25 | 25 |
| 7 | 7 | 14 | 14 | 14 | 14 | 14 | 14 | 14 | 14 | 14 | 14 | 14 |
| 60 | 65 | 35 | 40 | 45 | 50 | 55 | 60 | 65 | 70 | 75 | 40 | 45 |
| 25 | 25 | 30 | 30 | 30 | 30 | 30 | 30 | 30 | 30 | 30 | 35 | 35 |
| 14 | 14 | 14 | 14 | 14 | 14 | 14 | 14 | 14 | 14 | 14 | 14 | 14 |
| 50 | 55 | 60 | 65 | 70 | 75 | 45 | 50 | 55 | 60 | 65 | 70 | 75 |
| 35 | 35 | 35 | 35 | 35 | 35 | 40 | 40 | 40 | 40 | 40 | 40 | 40 |
| 14 | 14 | 14 | 14 | 14 | 14 | 14 | 14 | 14 | 14 | 14 | 14 | 14 |
| 50 | 55 | 60 | 65 | 70 | 75 | 55 | 60 | 65 | 70 | 75 | 60 | 65 |
| 45 | 45 | 45 | 45 | 45 | 45 | 50 | 50 | 50 | 50 | 50 | 55 | 55 |
| 14 | 14 | 14 | 14 | 14 | 14 | 14 | 14 | 21 | 21 | 21 | 21 | 21 |
| 70 | 75 | 65 | 70 | 75 | 70 | 75 | 75 | 60 | 65 | 70 | 65 | 70 |
| 55 | 55 | 60 | 60 | 60 | 65 | 65 | 70 | 55 | 55 | 55 | 60 | 60 |
| 21 | 21 | 21 | 21 | 21 | 21 | | | | | | | |
| 75 | 70 | 75 | 80 | 85 | 90 | | | | | | | |
| 60 | 65 | 70 | 75 | 80 | 85 | | | | | | | |

### Example 3

### Clinical Trial of MM-111

A first-in-human phase 1-2 study evaluates the safety and tolerability of MM-111 and preliminarily explores efficacy in HER-2+ advanced breast cancer (ABC). The safety data obtained during the Phase 1 dose escalation portion of this study provide the basis of this report.

Methods: Patients (pts) with histologically confirmed HER-2+ advanced solid tumors progressing or recurring on standard therapy; aged ≧18 years; ECOG PS <2 and adequate organ function were eligible for Phase I. Pts with stable CNS lesions were also eligible. A Modified Fibonacci schema was used for dose escalation in Phase I. Primary endpoints for Phase 1 were determination of maximum tolerated dose/maximum feasible dose while secondary endpoints included determination of dose-limiting toxicity, adverse events, and pharmacokinetic (PK) and immunogenicity profiles of MM-111. MM-111 was administered intravenously weekly in 4-week cycles.

### Example 4

### Clinical Trial Results—Overview

12 patients (11 ABC and 1 gastric cancer) were treated: median age 59 (range 36-82), median PS 1 (range 0-1), 11 ♀: 1♂, median number of prior therapies 7 (3-12). A total 19 courses (median 2; range 1-2) of therapy was administered at 4 dose-levels (3 mg/kg, 6 mg/kg, 12 mg/kg and 20 mg/kg respectively). Adverse events (see Example 3) assessed as being at least possibly related to MM-111 included pain (n=1), fatigue (n=3), dyspepsia (n=1), muscle spasms (n=1), heartburn (n=1), infusion reaction (n=1) and nail changes (n=1). There were no treatment interruptions due to adverse events. No dose limiting toxicities were observed and there has been no evidence of cardiotoxicity or immunogenicity to date. PK data indicate dose proportionality at the dose-levels explored and support weekly dosing.

### Example 5

### Clinical Trial Results—Safety and Pharmacokinetics

Patients (pts) with histologically confirmed HER-2+ advanced solid tumors progressing or recurring on standard therapy; aged >18 years; ECOG PS <2 and adequate organ function were eligible for Phase I. Pts with stable CNS lesions were also eligible. A Modified Fibonacci schema was used for dose escalation in Phase I. Primary endpoints for Phase 1 were determination of maximum tolerated dose/maximum feasible dose while secondary endpoints included determination of dose-limiting toxicity, adverse events, and pharmacokinetic (PK) and immunogenicity profiles of MM-111. MM-111 was administered intravenously weekly in 4-week cycles.

Primary objectives: Phase 1: To determine the Phase 2 dose based upon either the maximum tolerated dose (MTD) or the maximum feasible dose with HER2-positive solid tumors. Phase 2: To estimate Progression-Free Survival (PFS) in patients with HER2-positive breast cancer progressing on trastuzumab and/or lapatinib

Secondary objectives: To describe the dose limiting toxicity of MM-111

To determine the adverse event profile of MM-111

To determine the objective response rate of MM-111 in patients with HER2-positive breast cancer in Phase 2

To determine the Clinical Benefit Rate in Phase 2

To determine the pharmacokinetic (PK) parameters and immunogenicity of MM-111

Study Design:

Phase I: standard “3+3” design

4 dosing cohorts (Amended to include a 5th cohort with loading dose, ongoing)

Dose escalation decisions are made following a 4-week DLT evaluation period.

MTD defined as highest dose level in which a DLT is experienced by <2 patients in a cohort

Patients receive MM-111 weekly in four week cycles

Phase II: Dosing at MTD or optimal biologic dose

Adverse events during Phase 1 and Phase 2 will be graded according to CTCAE v3.0

Responses will be assessed according to RECIST v1.1

Eligibility:

Histologically confirmed advanced cancer that is HER2+

Non-measurable (Phase 1 only) or measurable or disease

ECOG Performance Status 0 or 1

Phase 2 only: Prior trastuzumab or lapatinib therapy with progressive disease on or after treatment

Stable CNS disease requiring no therapy is acceptable

Normal LVEF

Normal renal and hepatic function

TABLE 2
Results as of May 2011: Demographics

| | |
|---|---|
| N | 18 |
| Male/Female | 2/16 |
| Median Age (Range) | 62 (36-82) |
| Tumor types | Breast = 16 |
| | Gastric = 1 |
| | Lung = 1 |
| Median # prior systemic therapies (range) | 6 (2-9) |

TABLE 3
Results as of May 2011: Dose Levels

| Dose (mg/Kg) | Number of Patients |
|---|---|
| 3 mg/kg | 3 |
| 6 mg/kg | 3 |
| 12 mg/kg | 3 |
| 20 mg/kg | 3 |
| 25 mg/kg (L) | 6 |
| 20 mg/kg (M) | |

Summary of Adverse Events:

Twelve patients were enrolled at doses up to 20 mg/kg. Six patients were enrolled at a loading dose (L) of 25 mg/kg and a maintenance dose (M) of 20 mg/kg No DLTs have been identified and a Maximum Tolerated Dose has not been reached.

There were no apparent trends in AEs, lab values, ECGs vital signs or effect on cardiac ejection fraction in any of the cohorts, and overall the safety profile has been consistent with the patient population. Twelve patients were tested for the presence of anti-MM-111 and anti-HSA antibodies, and all samples were negative.

TABLE 4
Summary of Serious Adverse Events as of May 2011

| | Total N = 18 | # of Patients with |
|---|---|---|
| System Organ Class | # Patients | ≧ Grade 3 Event |
| Musculoskeletal and connective tissue | 10 | 1 |
| disorders | | |
| Gastrointestinal disorders | 8 | 1 |
| General disorders and administration | 10 | |
| site conditions | | |
| Metabolism and nutrition disorders | 7 | 1 |
| Infections and infestations | 3 | |
| Injury, poisoning and procedural | 3 | |
| complications | | |
| Neoplasms benign | 4 | |
| Nervous system disorders | 5 | |
| Investigations | 3 | |
| Ear and labyrinth disorders | 1 | |
| Blood and lymphatic system disorders | 3 | |
| Psychiatric disorders | 1 | |
| Renal and urinary disorders | 1 | |
| Respiratory | 4 | 1 |
| Skin and subcutaneous tissue disorders | 7 | |

TABLE 5
Summary of adverse events assessed as related to MM-111

| | | | Total N = 12 |
|---|---|---|---|
| Event Term | CTCAE Grade | Relationship | # Patients |
| Dyspepsia | 1 | Possible | 1 |
| Fatigue | 2 | Possible | 3 |
| Pain | 1 | Possible | 1 |
| Muscle Spasms | 1 | Possible | 1 |
| Heartburn | 1 | Possible | 1 |
| Infusion Reaction | 2 | Probable | 1 |
| Nail Changes | 1 | Definite | 1 |

TABLE 6
MM-111 Serum Concentration Levels

| | | Cmax | Tmax | T½ | |
|---|---|---|---|---|---|
| COHORT | Value | (ng/ml) | (H) | (H) | AUC ( ) |
| | N | 3 | 3 | 3 | 3 |
| 3 mg/kg | Median | 62.6 | 1.50 | 48.78 | 3269.05 |
| | Min; Max | 47.50: 67.10 | 1.50: 2.00 | 47.34: 71.18 | 3060.27: 4753.0 |
| | CV % | 17.4 | 17.3 | 24.0 | 25.0 |
| 6 mg/kg | Median | 129.00 | 2.00 | 88.75 | 9751.35 |
| | Min; Max | 93.3: 166.0 | 1.50: 2.00 | 53.50: 142.66 | 5453.63: 12002.1 [symbol] |
| | CV % | 28.1 | 15.7 | 47.3 | 36.7 |
| 12 mg/kg | Median | 288.00 | 2.00 | 100.39 | 20274.60 |
| | Min; Max | 184.0: 304.0 | 1.50: 2.00 | 97.95: 116.61 | 11597.80: 23785. [symbol] |
| | CV % | 25.2 | 15.7 | 9.7 | 33.8 |
| 20 mg/kg | Median | 348.00 | 4.00 | 104.34 | 38216.25 |
| | Min; Max | 344.0-522.0 | 1.5: 8.0 | 103.61-107.95 | 35136.75: 48518 [symbol] |
| | CV % | 25.1 | 72.9 | 2.2 | 17.3 |
| | N | 5 | 5 | 5 | 5 |
| 25 mg/kg | Median | 6.72E+05 | 1 | 90 | 7.69E+07 |
| | Min | 5.66E+05 | 1 | 84 | 6.44E+07 |
| | Max | 9.80E+05 | 4 | 155 | 1.51E+08 |

[symbol]

indicates data missing or illegible when filed

### Example 6

### Administration of MM-111 at Greater than Weekly Intervals

The foregoing results indicate that effective administration of MM-111 can be achieved with dosing at greater than weekly intervals.

Those skilled in the art will recognize, and will be able to ascertain and implement using no more than routine experimentation, many equivalents of the specific embodiments described herein. Such equivalents are intended to be encompassed by the following claims. Any combinations of the embodiments disclosed in the dependent claims are contemplated to be within the scope of the disclosure.

All publications, patent applications, and patents mentioned in this specification are herein incorporated by reference to the same extent as if each independent publication, patent application, or patent was specifically and individually indicated to be incorporated by reference. In particular, U.S. Ser. No. 61/421,992 in hereby incorporated by reference in its entirety.

From the foregoing description, one skilled in the art can easily ascertain the essential characteristics of this invention; can make various changes and modifications of the invention to adapt it to various usages and conditions. Thus, other embodiments are also within the claims.

1. A method for the treatment of a human patient diagnosed with a cancer characterized by a solid tumor that expresses ErbB2 receptor, the method comprising administering an effective amount MM-111 to the patient repeatedly at an interval measured in days, the administering comprising administering to the patient a single loading dose of at least 20 mg/kg of MM-111 followed at at least seven day intervals by at least one administration of a single maintenance dose of MM-111, wherein the maintenance dose is smaller than the loading dose.

2.-4. (canceled)

5. A method for the treatment of a human patient diagnosed with cancer characterized by a solid tumor that expresses ErbB2 receptor, the method comprising administering an effective amount MM-111 to the patient, the method comprising: administering to the patient an initial dose of at least 10 mg/kg of MM-111 followed by at least one administration of a subsequent dose of MM-111, each subsequent dose administered at an interval of at least seven days, which subsequent dose is the same as the initial dose.

6.-32. (canceled)

33. The method of claim 1, wherein the effective amount of MM-111 is administered by intravenous infusion.

34. The method of claim 33, wherein the maintenance dose is at least 5 mg/kg less than the loading dose.

35. The method of claim 33, wherein the at least seven day interval is a 14 day interval.

36. The method of claim 33, wherein the at least seven day interval is a 21 day interval.

37. The method of claim 33, wherein the at least seven day interval is an interval of a number of days indicated by a top number, the loading dose of at least 20 mg/kg of MM-111 is a dose in mg/kg indicated by a middle number, and the maintenance dose of MM-111 is a dose in mg/kg indicated by a bottom number in a cell of Table 1C selected from any populated cell of Table 1C other than the first two cells of the top row of Table 1 C.

38. The method of claim 5, wherein the effective amount of MM-111 is administered by intravenous infusion.

39. The method of claim 38, wherein the dose is about 15 mg/kg.

40. The method of claim 38, wherein the dose is about 20 mg/kg.

41. The method of claim 38, wherein the dose is about 25 mg/kg.

42. The method of claim 38, wherein the dose is about 30 mg/kg.

43. The method of claim 38, wherein the dose is about 35 mg/kg.

44. The method of claim 38, wherein the dose is about 40 mg/kg.

45. The method of claim 38, wherein the dose is about 45 mg/kg.

46. The method of claim 38, wherein the dose is about 50 mg/kg.

47. The method of claim 38, wherein the interval of at least seven days is an interval of a number of days indicated by a top number and the dose of at least 15 mg/kg of MM-111 is a dose in mg/kg indicated by a bottom number in a cell of Table 1C selected from any populated cell of Table 1C other than the first ten cells of the top row of Table 1 C.

48. The method of claim 38, wherein the interval of at least seven days is a 14 day interval.

49. The method of claim 38, wherein the interval of at least seven days is a 21 day interval.

50. The method of claim 38, wherein the at least one administration of a subsequent dose of MM-111 is at least two administrations.


SEQUENCE LISTING (1 sequence)

SEQ ID NO: 1 - protein, 1095 aa, Artificial Sequence
  Synthetic Construct
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG